(19) United States
(12) Patent Application Publication
(10) Pub. No.: US 2018/0133331 A1
(43) Pub. Date: May 17, 2018

(54) Site-Specific DNA-Doxorubicin Conjugates Display Enhanced Cytotoxicity to Breast Cancer Cells

(71) Applicants: William Henry Gmeiner, Yadkinville, NC (US); Freddie R. Salsbury, Winston Salem, NC (US); Mark O Lively, Winston Salem, NC (US); Chris H Stuart, Winston Salem, NC (US)

(72) Inventors: William Henry Gmeiner, Yadkinville, NC (US); Freddie R. Salsbury, Winston Salem, NC (US); Mark O Lively, Winston Salem, NC (US); Chris H Stuart, Winston Salem, NC (US)

(21) Appl. No.: 15/116,915

(22) PCT Filed: Feb. 5, 2015
(86) PCT No.: PCT/US15/14549

§ 371 Date: Jan. 11, 2018

Related U.S. Application Data
(60) Provisional application No. 61/936,028, filed on Feb. 5, 2014.

(51) Int. Cl. A61K 47/54; A61P 35/00; A61K 47/55; C12N 15/113; A61K 31/704

(52) U.S. Cl. CPC A61K 47/549; A61P 35/00; A61K 47/55; C12N 15/113; A61K 47/545; C12N 2310/336; C12N 2310/531; C12N 2320/31; C12N 2320/32; C12N 2310/351; A61K 31/704

(57) ABSTRACT

Doxorubicin (Dox) is widely used for breast cancer treatment but causes serious side-effects including cardiotoxicity that may adversely impact patient lifespan even if treatment is successful. The present invention relates to selective conjugation of Dox to a single site in a DNA hairpin resulting in a highly stable complex that enables Dox to be used more effectively. Selective conjugation of Dox to G15 in the hairpin loop was verified using site-specific labeling with [2-15N]-2′-deoxyguanosine in conjunction with [1H-15N] 2D NMR while 1:1 stoichiometry for the conjugate was validated by ESI-QTOF mass spectrometry and UV spectroscopy. Molecular modeling indicated covalently bound Dox also intercalated into the stem of the hairpin and stability studies demonstrated the resulting Dox-conjugated hairpin (DCH) complex had a half-life >30 h, considerably longer than alternative covalent and non-covalent complexes. Secondary conjugation of DCH with folic acid (FA) resulted in increased internalization into breast cancer cells. The dual conjugate, DCH-FA, can be used for safer and more effective chemotherapy with Dox and this conjugation strategy can be expanded to include additional anti-cancer drugs.

### STATEMENT REGARDING FEDERALLY SPONSORED RESEARCH OR DEVELOPMENT

The present invention claims priority under 35 USC 119(e) to U.S. Provisional Application No. 61/936,028 filed Feb. 5, 2014, the entire contents of which are incorporated by reference in its entirety.

The present invention was supported by Department of Defense Prostate Cancer Research Program (093606) and with funds from NIH Shared instrumentation Grant 1S10RR17846. The present invention was also supported in part by NCI center grant 5P30CA 12197. Accordingly, the federal government has rights in the present invention.

### BACKGROUND OF THE INVENTION

Doxorubicin (Dox) is widely-used for treating breast cancer and other malignancies. However serious toxicities, including an occasionally lethal cardiotoxicity, counter the therapeutic benefit of Dox resulting in a search for chemical modifications that attenuate systemic toxicities while maintaining strong anti-tumor activity. The principal cytotoxic mechanism of Dox is poisoning of DNA topoisomerase 2 (Top2) which results in generation of lethal DNA double strand breaks (DSBs). Dox also undergoes REDOX cycling and increases oxidative stress following cell uptake. Some have suggested Dox cardiotoxicity results from an on-target effect, the poisoning of Top 2 in cardiomyocytes. Hence, it is thought that strategies to improve the therapeutic index of Dox require prolonged sequestration of Dox while in circulation and efficient Dox release following selective uptake into targeted cancer cells.

DNA is central to biological function as the repository of genetic information but DNA also has tremendous potential as a material with diverse potential functions, including drug delivery. Previously, the utility of DNA for delivery of cytotoxic nucleotide analogs has been shown. For example, DNA delivery with F10, a polymer of the thymidylate synthase (TS) inhibitory nucleotide 5-fluoro-2‘-deoxyuridine-S’-O-monophosphate (FdUMP) displayed enhanced anti-leukemic activity and reduced systemic toxicity relative to conventional fluoropyrimidine drugs such as 5-fluorouracil (5-FU). Dox interacts with DNA via intercalation of the tetracene ring system between the planar base pairs of duplex DNA and occupation of the minor groove by the daunosamine sugar moiety. Non-covalent binding of Dox to DNA is however readily reversible and non-covalent complexes have relatively short half-lives (t1/2˜minutes). Nonetheless, in clinical trials non-covalent association of Dox with calf-thymus DNA reduced Dox cardiotoxicity and improved the therapeutic index. Dox also forms covalent adducts with DNA that are more stable but require an aldehyde precursor to link the daunosamine sugar of Dox to the exocyclic amine of guanine with the reaction proceeding via a Schiff base intermediate. Dox:DNA covalent adducts are more cytotoxic than non-covalent complexes and covalent adducts have been synthesized and used as end-points in studies of anthracycline cytotoxicity. Formaldehyde is used in formation of Dox-DNA adducts and exogenous formaldehyde promotes Dox covalent adduct formation to genomic DNA. Dox-formaldehyde conjugates have been prepared and used for delivery of an activated form of Dox that favors covalent adduct formation to genomic DNA.

### BRIEF SUMMARY OF THE INVENTION

In an embodiment, the present invention relates to a new approach for Dox delivery to cancer cells that takes advantage of the selective chemical reactivity of a single-site in a DNA hairpin to create a novel Dox-conjugated DNA hairpin (DCH) with favorable Dox retention and release properties and that is targeted to breast cancer cells via folic acid conjugation.

In an embodiment, the present invention relates to Dox-hairpins using anti-folates that target one or more of folate receptor alpha and beta (e.g. lomotrexol, pemetrexed). In a variation, the present invention relates to conjugates, compositions and methods that may also use reduced folate carrier (RFC) e.g. methotrexate. It should be noted that RFC is expressed widely so may have applications in a more general manner while FRα and FRβ are cancer cell specific and thus, may be used to more directly target cancer cells. FRα may be more important as it relates to solid tumors. In one embodiment, the hairpin has a unique adenine site that can be used for the anti-folate targeting—for example, A13 (adenine at the 13′ position of the DNA hairpin structure) from SEQ ID NO: 1 (see below).

In an embodiment, the present invention also relates to using DNA hairpins for drug delivery with involvement of both the major and minor grooves as well as the duplex region of the hairpin. Cytotoxicity can be modulated by inclusion of minor groove binding ligands, such as netropsin or distamycin, while Zn2+, a metal ion that displays anti-cancer activity, can occupy the major groove in DNA hairpins appropriately substituted with FdU nucleotides in the stem. Hence, not only are the chemical properties of DNA of potential use for drug delivery but its structural diversity may also be utilized for drug delivery applications.

In an embodiment, the present invention also relates to the synthesis of a covalent conjugate of Dox to a single site of a DNA hairpin. In an embodiment, the present invention demonstrates this conjugate can be targeted to breast cancer cells. Dox covalent binding to DNA occurs primarily at N2 of guanines with sequence specificity for 5′-dGpC sites, suggesting a 3D conformation that facilitates covalent binding. In a variation, the present invention utilizes a 25mer DNA hairpin that includes a GAA hairpin-promoting sequence closed by a CG base pair with the stem consisting of 10 dA-dT base pairs (see FIG. 1). Although the hairpin includes two dG sites, using 2D NMR in conjunction with site-specific labeling it was determined that only G15 in the GAA hairpin promoting motif formed a covalent adduct with Dox (see FIG. 2). Molecular modeling suggested G15 N2 was not engaged in alternative interactions stabilizing the hairpin and that the tetracene ring system intercalated between the CG and first AT base pairs (see FIG. 3). The Dox-conjugated hairpin was exceedingly stable with a half-life of ˜30 h at physiological pH while the non-covalent complex had a half-life of minutes (see FIG. 4). Dox was however efficiently released at the acidic pH of endosomes following cell uptake (see FIG. 5). Folic acid conjugation of the hairpin resulted in cell-specific uptake into breast cancer cells and selective cytotoxicity towards targeted cells (see FIG. 6). These results demonstrate the utility of DNA hairpin conjugates for the improved delivery of Dox and other anti-cancer drugs.

### BRIEF DESCRIPTION OF THE SEVERAL VIEWS OF THE DRAWING

FIG. 1 shows a reversible reaction between Doxorubicin and the exocyclic amino of Guanine in DNA mediated through formaldehyde. The “G” in the secondary structure of the hairpin denotes potential sites of Dox reactivity.

FIGS. 2 A, B, and C show A) UV-Vis absorbance spectra for equimolar amounts of Dox and DCH's. Equal absorbance at 480 nm is consistent with the DCH complex being of 1:1 stoichiometry B) M-Dox peak of MW 7727 corresponds to the un-reacted parent DNA, while M+ peak of MW 8283 is obtained by the addition of Dox (MW 543) and CH₂ (MW 14)—2H lost as water. C) Overlay of ¹H-¹⁵N HSQC displaying the N2 of G12 and G15 from two independent singly-labeled samples. Blue and green peaks represent the G12 amino before and after reaction with Dox, respectively. Black and red peaks represent the G15 amino before and after reaction respectively.

FIG. 3 shows Secondary structure and molecular model of the DCH. Dox is bound to N2 of G12 and intercalated between the G15:C11 and A10:T16 base pairs. The lower 9 AT base pairs of the stem have been truncated for simplicity. Structures are colored as follows; guanine:green, adenine:red, thymine:yellow, cytosine:blue, Dox:light blue, methylene linker:white, DNA backbone:brown A-C) 3D modeling of the DCH structure D) 3D model of the unreacted hairpin.

FIG. 4 shows Fluorescence quenching of DCH that displays a≈50% reduction in fluorescence after 30 hours while non-covalent complexes display greater than 50% reduction in fluorescence within 1 hour (data not shown). Error bars represent standard deviation of the mean of three measurements. Assuming zero order kinetics, the rate constant is k=1.15×10⁻¹¹ M/s

FIGS. 5 A and B show A) Fluorescence microscopy of 4T1 cells treated with either untargeted or folate-targeted DCH. B) Quantification of Dox fluorescence from 4T1 cells. Error bars represent standard deviation from the mean of at least 30 measurements. A student's two-tailed t-test was used to determine significance.

FIG. 6 shows targeting DCH with folic acid (DCH-FA) significantly increases the cytotoxicity of the DCH construct towards 4T1 breast cancer cells. Error bars represent standard deviation from the mean with four replicates of each condition. A student's two-tailed t-test was used to determine significance (i.e. p<0.05)—*significantly different from control; ^{Δ}significantly different from DCH-FA.

### DETAILED DESCRIPTION OF THE INVENTION

The present invention relates to DNA hairpin conjugates, compositions, and methods thereof for the improved delivery of anti-cancer drugs to individuals that are in need thereof. In an embodiment, the conjugates compositions and methods thereof relate to the improved delivery of Dox to individuals in need thereof.

In an embodiment, the present invention relates to conjugating anti-cancer drugs, and for example, the Dox-hairpin with folic acid to individuals that have cancer. In one embodiment, the present invention relates to attaching the folic acid at the 5′-terminus of the Dox-hairpin. In an embodiment, the conjugate with (or without) folic acid can be used in the uptake into breast cancer cells. In a variation, the present invention relates to the conjugate, composition, and associated method that can be used in 4T1 breast cancer cells.

In an embodiment, the present invention relates to a new approach for Dox delivery to cancer cells that takes advantage of the selective chemical reactivity of a single-site in a DNA hairpin to create a novel Dox-conjugated DNA hairpin (DCH) with favorable Dox retention and release properties and that is targeted to breast cancer cells via folic acid conjugation.

In an embodiment, the present invention relates to Dox-hairpins using anti-folates that target one or more of folate receptor alpha and beta (e.g. lomotrexol, pemetrexed). In a variation, the present invention relates to conjugates, compositions and methods that may also use reduced folate carrier (RFC) e.g. methotrexate. It should be noted that RFC is expressed widely so may have applications in a more general manner while FRα and FRβ tend to be cancer cell specific and thus, may be used to more directly target cancer cells. FRα is likely to have uses particularly as they relate to solid tumors. In one embodiment, the hairpin has a unique site that can be used for the anti-folate targeting A13.

In an embodiment, the present invention also relates to using DNA hairpins for drug delivery with involvement of both the major and minor grooves as well as the duplex region of the hairpin. Cytotoxicity can be modulated by inclusion of minor groove binding ligands, such as netropsin or distamycin, while Zn2+, a metal ion that displays anti-cancer activity, can occupy the major groove in DNA hairpins appropriately substituted with FdU nucleotides in the stem. Hence, not only are the chemical properties of DNA of potential use for drug delivery but its structural diversity may also be utilized for drug delivery applications.

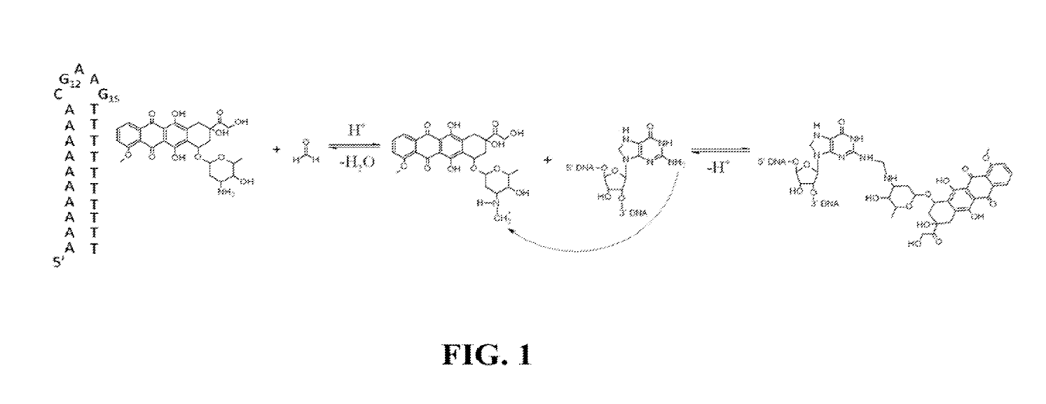

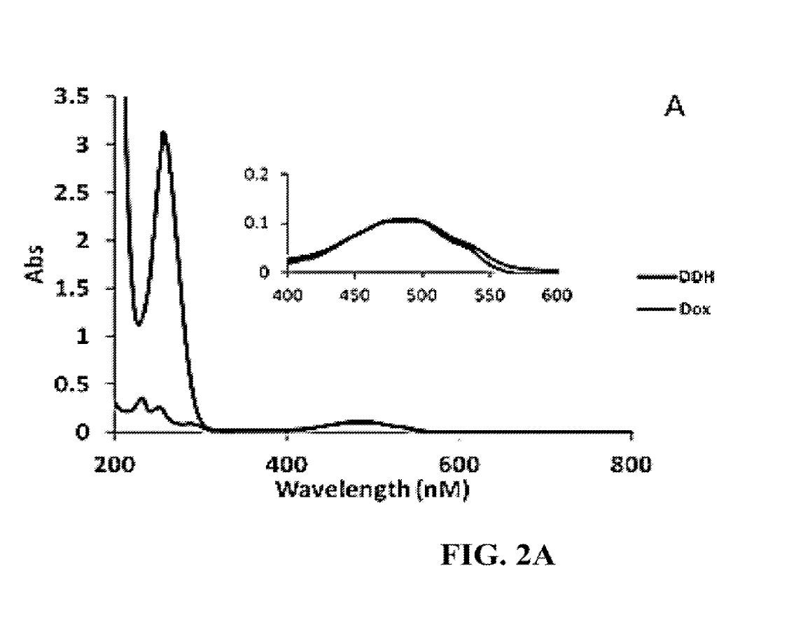

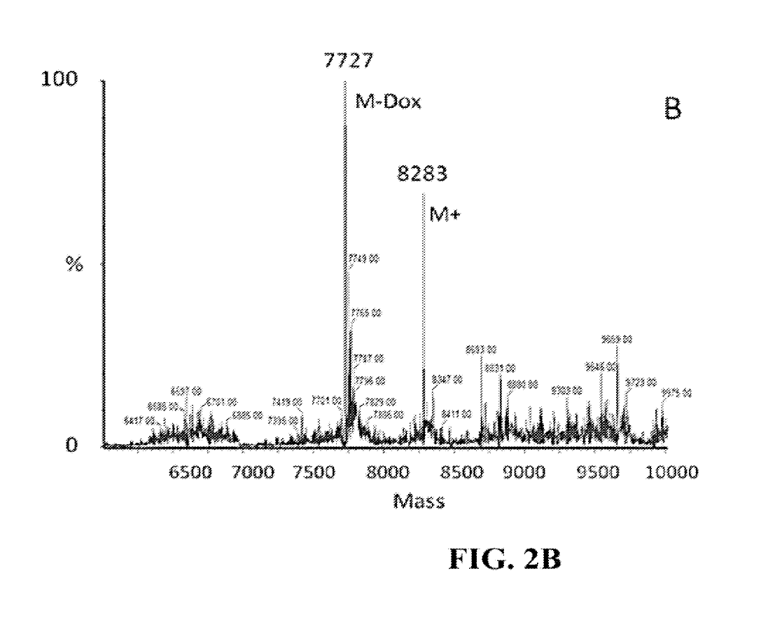

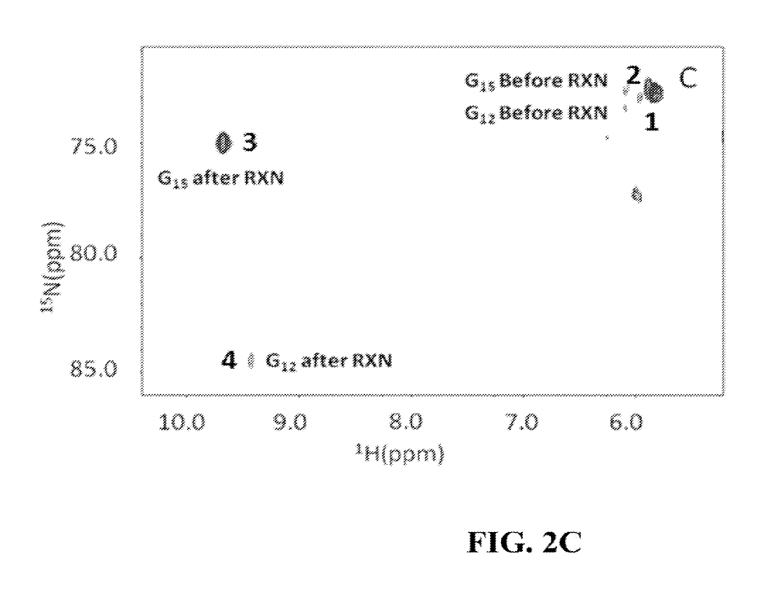

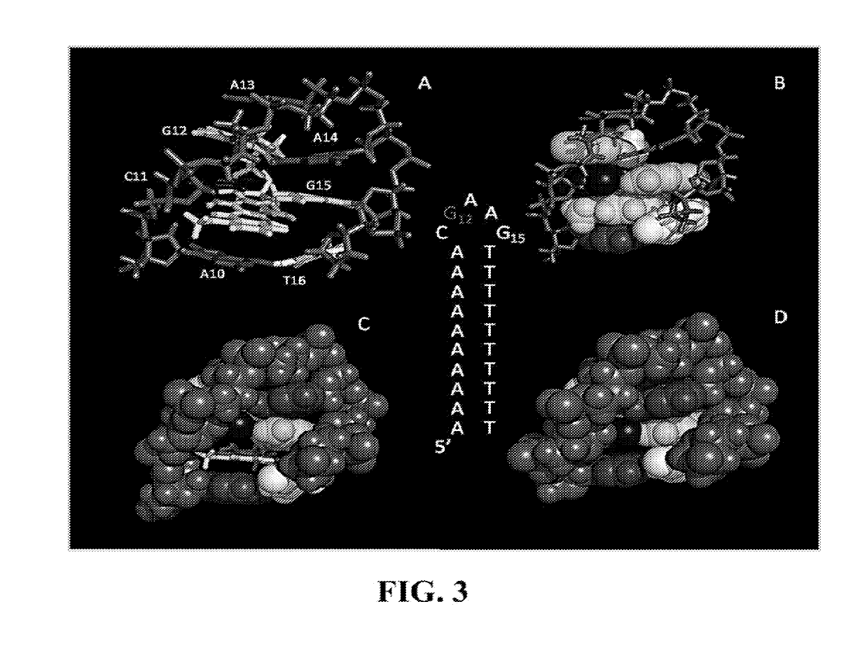

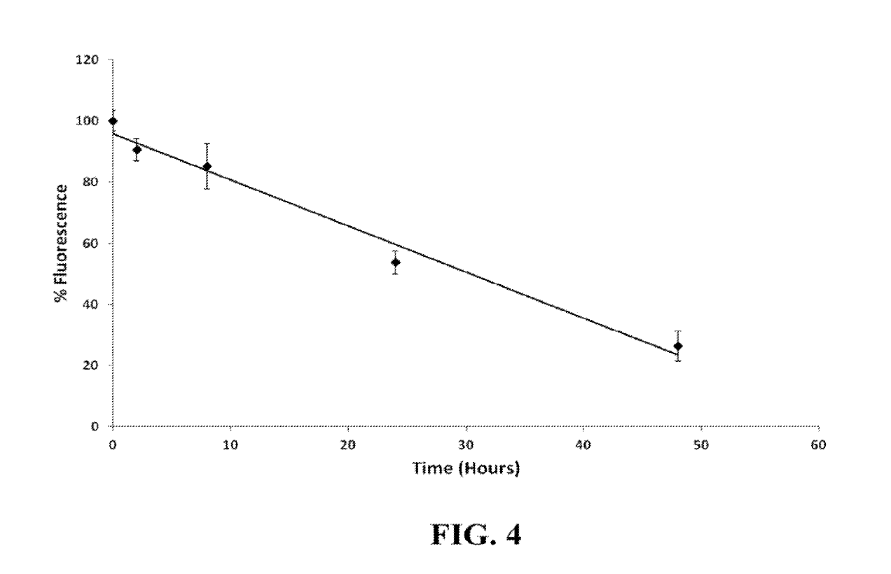

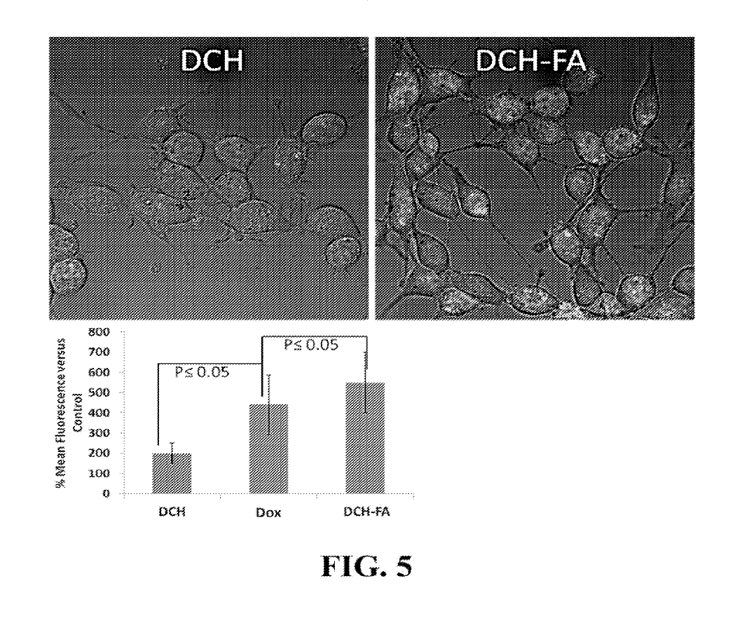

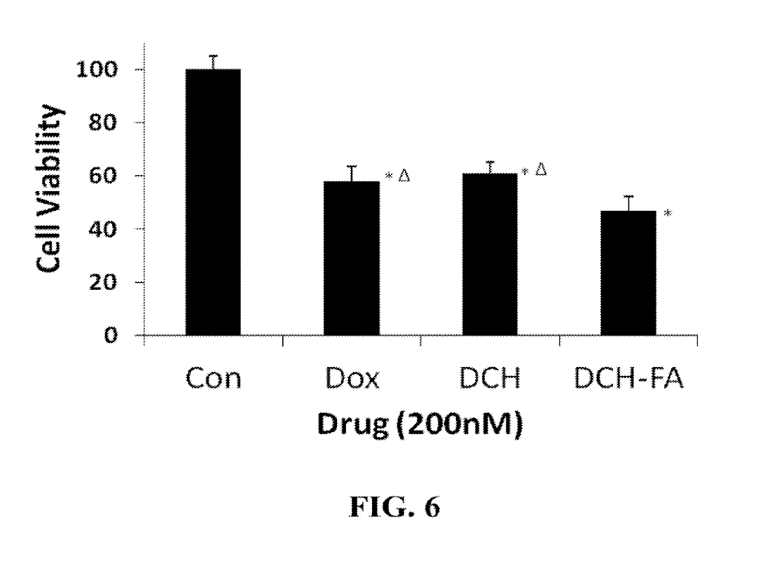

In an embodiment, the present invention also relates to the synthesis of a covalent conjugate of Dox to a single site of a DNA hairpin. In an embodiment, the present invention demonstrates this conjugate can be targeted to breast cancer cells. Dox covalent binding to DNA occurs primarily at N2 of guanines with sequence specificity for 5′-dGpC sites, suggesting a 3D conformation that facilitates covalent binding. In a variation, the present invention utilizes a 25mer DNA hairpin that includes a GAA hairpin-promoting sequence closed by a CG base pair with the stem consisting of 10 dA-dT base pairs (see FIG. 1). Although the hairpin includes two dG sites, using 2D NMR in conjunction with site-specific labeling it was determined that only G15 in the GAA hairpin promoting motif formed a covalent adduct with Dox (see FIG. 2). Molecular modeling suggested G15 N2 was not engaged in alternative interactions stabilizing the hairpin and that the tetracene ring system intercalated between the CG and first AT base pairs (see FIG. 3). The Dox-conjugated hairpin was exceedingly stable with a half-life of ˜30 h at physiological pH while the non-covalent complex had a half-life of minutes (see FIG. 4). Dox was however efficiently released at the acidic pH of endosomes following cell uptake (see FIG. 5). Folic acid conjugation of the hairpin resulted in cell-specific uptake into breast cancer cells and selective cytotoxicity towards targeted cells (see FIG. 6). These results demonstrate the utility of DNA hairpin conjugates for the improved delivery of Dox and other anti-cancer drugs.

The following examples are exemplary of the drug conjugates that can be made and delivered to individuals in need thereof. Although the examples given below are specific, it should be recognized by those of ordinary skill in the art, that the conjugates, compositions, and associated methodologies can be generalized to other anti-cancer drugs.

Materials: All non-labeled hairpin DNA sequences were synthesized by IDT (Coralville Iowa, USA). Isotopically-labeled hairpins were synthesized by the DNA core lab at Wake Forest University. Clinical samples of doxorubicin (Dox) used for cell assays and DCH synthesis were obtained from the Wake Forest Baptist hospital pharmacy. Cu2+-Tris[(1-benzyl-1H-1,2,3-triazol-4-yl)methyl]amine (Cu2+-TBTA) was obtained from Lumiprobe (Hallandale Beach Fla., USA). All media for cell culture was obtained from the Wake Forest University Cell and Viral Vector Core Lab. All other chemicals were obtained from Sigma-Aldrich and used as received.

Hairpin DNA sequence 5′-AAAAAAAAAACGAAGTTTTTTTTTT-3′ (SEQ ID NO. 1) was synthesized as described above.

Synthesis of DCH: A 0.37% (by weight) solution of formaldehyde was prepared by dissolving paraformaldehyde in Dulbecco's Phosphate Buffered Saline without Calcium or Magnesium (PBS) pH 7.4. Doxorubicin was added to 4° C. formaldehyde-PBS to obtain a final doxorubicin concentration of 250 μM. DNA hairpin loops were prepared as previously reported6 by heating and flash-cooling of the DNA to favor intramolecular hairpin formation over dimerization. Hairpins were added to the Dox-formaldehyde solution to obtain a final hairpin concentration of 100 μM. Reactions were allowed to proceed at 10° C. in the dark for 48 hours. DCH's were purified by extracting twice with phenol:chloroform and twice with chloroform. This extraction removes unreacted dox from the solution. After extraction, DCH's were ethanol-precipitated and recovered by centrifugation. Pellets were rinsed twice with 70% ethanol and 100% ethanol to remove any residual formaldehyde. Pellets were then evaporated to dryness under reduced pressure. The red-pink pellets were then re-suspended in water. A Beckman Coulter DU 800 was used to measure absorption at 260 nm. Yields were typically 70-80% for the conjugate as measured by UV absorbance at 260 nm. All products were stored at −20° C.

Synthesis of alkyne functionalized folic acid: Alkyne functionalized folic acid was synthesized similarly to previously reported methods. Briefly, folic acid (100 mg, 0.227 mmol) was dissolved into 10 mL of DMF and stirred with a magnetic stirrer and cooled in an ice bath for 30 minutes before proceeding. 1-Ethyl-3-(3-dimethylaminopropyl)carbodiimide HCl (EDC) (38.7 mg, 0.249 mmol) was added to the stirred solution and allowed to react for 30 minutes. N-Hydroxysuccinimide (NHS) (31.4, 0.272 mmol) was then added to the reaction vessel and stirred for an additional 30 minutes. Propargyl amine (25 mg, 0.454 mmol) was then added to the reaction, which was warmed to room temperature and allowed to react for 24 hours. 10 mL of 1:1 diethyl ether:chloroform was added to the reaction vessel which precipitated the yellow-orange product. This was collected and washed three times with chloroform, diethyl ether, and water. The product was dried under vacuum overnight. Yield 69 mg (64%). 1H NMR (DMSO-d6, ppm): 11.06 (—OH), 8.64 (PteridineC7 H), 8.29-8.24 (—CONH—CH2C≡CH), 8.04 (—CONHCHCO2H), 7.67-7.65 (Ph-C2H and Ph-C6H), 6.94 (—NH2), 6.64 (Ph-C3H and Ph-C5H), 4.48 (PteridineC6-CH2NH-Ph), 4.31 (—CONHCHCO2H), 3.81 (—CONH—CH2C≡CH), 3.08 (—CONH—CH2C≡CH), 1.98-1.96 (—CHCH2CH2), 1.87-1.85 (—CHCH2CH2, 2H).

Synthesis of FA-DCH: Hairpin with a 5′ terminal azide was used for the synthesis of FA-DCH. 45 μL of a 100 μM solution of the hairpin was added to a 500 μL centrifuge tube. To this 10 μL (pH=7, 2M) of triethylamine/acetic acid buffer was added and mixed. 45 μL DMSO was then added to the solution and mixed well. 4.5 μL of a 10 mM solution of alkyne functionalized folic acid was added to the solution, mixed, and bubbled with Argon for 15 minutes. 10 μL of 5 mM ascorbic acid in water was added to the solution followed by 5 μL of Cu2+-TBTA. The reaction was mixed and bubbled with Ar for 15 minutes before sealing the tube and being placed in the dark at room temperature for 24 hours. FA-DCH was purified similarly to DCH. The hairpin was precipitated using 4× volume ethanol and 25 μL PBS and was cooled at −20° C. for 30 minutes. The hairpin was recovered by centrifugation at 13000×G for 30 minutes. The pellet was rinsed 2× with 70% EtOH and 2× with 100% EtOH and dried under reduced pressure. The pellet was re-suspended with 500 μL PBS and was dialyzed against PBS using a Slide-a-Lyzer 2 kDa MW cutoff dialysis cassette (Pierce) for 6 hours to remove unreacted propargyl-folate. The retained solution was collected and quantified via UV-Vis spectroscopy, with a final yield of 53%.

Doxorubicin:DNA conjugate ratio measurements: DNA samples were prepared to 10 μM in dH2O and absorbencies were measured from 200-800 nm using a Beckman Coulter DU800 spectrophotometer. A standard curve of Dox was established between 1 μM and 10 μM by using absorbance at 494 nm. To assess the amount of Dox covalently bound to DNA the samples were heated to 85° C. before measuring the absorbance at 494 nm. The 260 nm wavelength was used to determine the DNA content in the sample and to determine the Dox:DNA ratio.

Mass Spectrometry: Negative ion mass spectra were acquired using a Waters Q-TOF API-US mass spectrometer equipped with an Advion Nanomate source. Samples were diluted to about 5 μM with methanol:water:2-propanol (49:49:2, v:v:v). Backing pressure and sprayer voltage were optimized for each analysis, but were usually about 0.8 psi and 1.2 kV, respectively. The cone voltage was 35V. The scan range from 525 m/z to 1600 m/z with an acquisition time of 1.2 s. Spectra were summed for 0.5 min for MaxEnt transform. The nucleotide GCATCCTGGAAAGCTACCTT, M−=6366.1, at 0.6 μM was used to monitor instrument performance. Spectra were analyzed using MassLynx 4.0.

NMR Spectroscopy: NMR samples were prepared in 50 mM sodium phosphate buffer, pH 7.0, with 10% D20 and a final volume of 250 μL All NMR spectra were acquired using a Bruker Avance 600 MHz spectrometer at 10° C. using a TXI Cryoprobe. NOESY spectra were acquired with a 100 μs mixing time and 3-9-19 Watergate water suppression with a 220 ms interpulse delay. HSQC spectra were acquired using a 110 μs 3-9-19 interpulse delay and the 15N transmitter set to 150 PPM for imino groups and to 75 PPM for amino groups (indirectly referenced to water at 4.7 PPM). Data were processed using NMRPipe and analyzed using NMRView.

3D modeling of DCH: A PDB file of the hairpin molecule was obtained from the Protein Data Base under entry 1JVE. A PDB file of Dox was obtained from the Protein Data Base under entry DM2. Files were loaded into Pymol, and the hairpin was modified to contain only 5′-ACGAAGT-3′. The models were then manipulated spatially to allow for a covalent bond to form between the N2 amino of G12 and the daunosamine of Dox. Hydrogens were added to the entire model using the molefacture plug in vmd. The doxorubicin was then geometry optimized in the presence of the DNA using PM6 as implemented in Gaussian 09.

Dox transfer from DCH: Samples of 2.5 μM (approximately 2 μg in 100 uL) DCH, doxorubicin, or hairpin+doxorubicin were prepared in DPBS with or without a 100-fold by weight (200 μg) excess of Salmon Sperm DNA and incubated at 37° C. Fluorescence intensity was determined by Typhoon-9210 variable mode imager with excitation set to 532 nm and the emission filter at 610 nm.

Acid dissociation of Dox from DCH: DCH was suspended in either pH 7.4 PBS or pH 4 PBS buffer and incubated at 37° C. for 1 hour. After incubation the solutions were extracted with 2× volume phenol:chloroform and twice with 2× volume chloroform. The absorbance of the aqueous phase at 498 nm was measured. The experiment was repeated in triplicate. The results were normalized to the pH 7.4 sample with error bars representing the standard deviation of the mean of the three replicates.

Microscopy: 4T1 cells were seeded at 20,000 cells/well in 8-well Lab-Tek II chambered #1.5 German Coverglass System (Thermo Fisher Scientific, Waltham, Mass.), and incubated at 37° C. under 5% CO2 for 24 hours prior to treatment. Cells were incubated with 1 μM of DCH, FA-DCH, or Dox in DMEM medium with 10% dialyzed fetal bovine serum for either 1 or 4 hours at 37° C. Cells were then washed with fresh media and Dulbecco's PBS. Cells were visualized using a Zeiss LSM 510 confocal microscope (Carl Zeiss, Oberkochen, Germany) using Dox as the fluorescent probe. Internalization of Dox was quantified using ImageJ software with at least 30 observations per treatment. Fluorescence intensity values were then converted to % controls using non-treated cells. The mean of the intensities was found and standard deviation was determined. Significance was determined using a two-tailed student's t-test

Cytotoxicity: 4T1 cells were grown in DMEM media containing 10% dialyzed FBS and 1% penicillin/Streptomycin, at 37° C. and 5% CO2. 4T1 cells were plated at 5,000 cell per well in 96 well plates in 100 μL media and incubated for 24 hours. Cells were treated with 200 nM of DCH, Dox, or FA-DCH for 72 hours with 4 replicates of each treatment used to determine means and standard deviation. CellTiter-Glo luminescent cell viability assay (Promega) was implemented according to the manufacturer's protocol. Significance was determined using a two-tailed student's t-test.

### Results

Site-Specific Dox Conjugation of a DNA Hairpin. In one embodiment, the DNA hairpin used for these studies includes two guanines (see FIG. 1) either of which may in principle be a site for Dox conjugation. Dox conjugates have been previously described for guanines engaged in GC base pairs however the chemical reactivity of the GAA sequence motif used to promote intramolecular hairpin formation has not been previously investigated. In one embodiment, the present invention relates to UV spectroscopy studies (see FIG. 2A) that revealed Dox conjugation occurred with 1:1 stoichiometry even in cases where reaction conditions permitted formation of conjugates of 2:1 or higher stoichiometry. The 1:1 stoichiometry of the conjugates was further demonstrated using ESI-QTOF mass spectrometry (FIG. 2B). Mass spectrometry analysis also confirmed that the conjugation occurred via a methylene bridge derived from formaldehyde consistent with a reaction proceeding via a Schiff base intermediate. To determine to what extent each of the two guanines in the hairpin were adducted in the conjugate the hairpin site-was synthesized specifically with 15N-labeled at either G12 or G15 and allowed to form Dox conjugates with both species. The conjugates were analyzed each for chemical adduction based on chemical shift changes in 2D [1H-15N] NMR spectra (see FIG. 2C). Substantial 15N chemical shift changes were only detected for the hairpin labeled at G15 (72.6→84.5 ppm), consistent with this site selectively undergoing chemical modification upon adduct formation. This represents the first time that the inventors are aware of that Dox covalent bonding has been observed in a hairpin loop region of DNA. G12 which is engaged in a GC base pair that closes the loop underwent substantial change in 1H (5.8→9.7 ppm) but not 15N chemical shift (72.8→75.0 ppm) consistent with this site undergoing changes in chemical environment, but not chemical structure, upon Dox conjugation. Subsequent molecular modeling studies revealed the CG based pair was stacked with the tetracene ring system of Dox in the resulting conjugate (see infra).

Molecular Model of Dox-Conjugated Hairpin. A working model for the structure of the Dox-conjugated hairpin (FIG. 3) was created using the data collected from the 15N-edited 2D NMR and using a NMR structure of the hairpin loop as the starting point for model development. The hydrogen shifts of G15 could be caused by intercalation of Dox in the covalent complex, as shifts of amino protons to ≈10 ppm have been attributed to increased hydrogen bonding in quadruplex DNA. Preliminary data also showed the amino of G15 displayed several NOEs consistent with Dox localizing in the stem region of the hairpin. Pymol was used to edit the DNA from the previous NMR structure to contain only the loop region and the first AT base pair. A model of Dox was manipulated into a position that brought the amino hydrogens of G12 and Dox into close proximity, allowing for the formation of a methylene bridge between the amino nitrogens. This placement also allowed Dox to intercalate between the C11-G15 base pair and the first AT base pair in the stem of the DNA (see FIGS. 3A,B). Modeling revealed that the simultaneous covalent binding at G12 and intercalation of Dox between the A10:T16 and C11:G15 occurred with minimal distortion to the structure of the hairpin loop (FIG. 3C,D). The daunosamine sugar is of appropriate dimensions to span the distance between the sites of covalent binding and intercalation and the amino group of G12 is not engaged in hydrogen bonding interactions that contribute significantly to the structure and stability of the hairpin loop. Intercalation of the tetracene ring alters local base stacking for the proximal base pairs but does not disrupt hydrogen bonding interactions (FIG. 3D).

Stability of Dox:Hairpin Conjugate. It was hypothesized that covalent binding of Dox would allow for the Dox-conjugated hairpin to serve as a delivery vehicle with improved pharmacological properties and reduced systemic toxicities relative to conventional Dox. In order for the hairpin to act as an efficient delivery vehicle, Dox must remain stably bound under physiological conditions, but also undergo intracellular release and transfer to genomic DNA. Interestingly, Dox retains most of its fluorescent activity in the hairpin, but not in larger DNA molecules which are known to quench the fluorescence of the drug It was hypothesized that as the Dox-hairpin bond is hydrolyzed, the free Dox could intercalate into larger DNA molecules, quenching the fluorescence. Using the difference in fluorescence between hairpin-bound Dox and Dox intercalated into DNA, the half-life of the Dox-hairpin bond was measured. An assay was developed to quantify the transfer of Dox from the hairpin to genomic DNA under physiologic conditions. This assay is based on the difference in fluorescence of Dox in the context of the hairpin conjugate relative to genomic DNA. The hairpin conjugate, free-Dox, or the non-covalent complex (e.g. hairpin+Dox) were mixed with 100-fold excess of salmon sperm DNA (spDNA; w/w) to simulate genomic DNA. Reactions were incubated at 37° C. with fluorescence quenching measured over 48 h. No fluorescence loss was observed in samples that lacked spDNA, while fluorescence was fully quenched within one hour following addition of spDNA for both free Dox and the non-covalent complex. The rate of loss of fluorescence quenching was however significantly reduced for the hairpin conjugate with 50% quenching occurring at 30.4 h (see FIG. 4). Using t1/2 of 30.4 hr and assuming zero order kinetics, the rate constant is 1.15×10⁻¹¹ M/s. Given that the non-covalent hairpin+Dox complex undergoes rapid quenching, intercalation into the hairpin cannot be solely responsible for the increased chemical stability of the hairpin conjugate. Covalent Dox dimers formed using formaldehyde have been shown to be readily hydrolysable under physiological conditions, resulting in complete disassociation of Dox and formaldehyde release within ≈15 min. Thus, it is likely a combination of both intercalation and covalent bonding that is responsible for the substantially increased stability for the Dox-conjugated hairpin relative to the non-covalent complex and alternative Dox covalent complexes. It is believed that the covalent linkage acts as a tether between the Dox and DNA, and that when the bond is hydrolyzed intercalation holds the resulting amine and Schiff-base in close proximity allowing for them to reform the covalent linkage. At physiological pH equilibrium favors re-forming the covalent complex while at acidic pH, for example in endosomes, equilibrium disfavors re-forming the covalent linkage and instead results in release of Dox from the intercalated complex.

Targeted Uptake and Enhanced Cytotoxicity to Breast Cancer Cells. Cellular uptake of exogenous DNA can be highly efficient if uptake occurs via receptor-mediated processes. Initial studies with the Dox-conjugated hairpin indicated uptake into breast cancer cells was less efficient than for F10, a single-stranded DNA investigated in previous studies. As previous studies demonstrated that conjugation with folic acid improved F10 uptake into drug-resistant colon cancer cells it was investigated whether conjugating the Dox-hairpin at the 5′-terminus with folic acid would improve uptake into 4T1 breast cancer cells. Folic acid conjugation of the hairpin resulted in significantly increased cellular uptake relative to the non-conjugated hairpin based upon increased Dox fluorescence into 4T1 breast cancer cells (see FIG. 5). Dox fluorescence was initially localized in endosomes consistent with cellular internalization via an endocytic process and with release of Dox at the acidic pH of endosomes. Folic acid conjugation also increased the cytotoxicity of the Dox-conjugated hairpin towards 4T1 cells consistent with both improved cell uptake and efficient Dox release (FIG. 6). The results demonstrate that while the Dox-conjugated hairpin has markedly improved stability at physiological pH relative to the corresponding non-covalent complex the conjugate is highly effective at the intracellular release of Dox following cell uptake.

DNA is central to biology as predominant carrier of genetic information however the physical and chemical properties of DNA make it highly useful as a material for numerous applications including use for drug-delivery. These studies have demonstrated that a simple DNA hairpin that includes a “GAA” hairpin-promoting sequence provides a unique site for conjugation with the Top2-poisoning anti-cancer drug Dox. Conjugation occurs without disrupting stabilizing hydrogen bonding or base stacking interactions in the hairpin loop and allows for facile intercalation of the tetracene ring system of Dox between the first and second base pairs of the hairpin stem. The concurrent covalent linkage and intercalation of Dox in the hairpin results in formation of a complex that is highly stable at physiological pH. As non-covalent Dox:DNA complexes, presumably of greatly reduced chemical stability relative to the Dox-conjugated DNA hairpin described here have shown decreased toxicity relative to free Dox in human clinical trials hairpin conjugates may represent an improved approach for limiting Dox toxicity while preserving Dox efficacy.

A number of approaches have been described for improved Dox delivery while limiting systemic toxicities. The liposomal formulation Doxil, for example, has demonstrated clinical utility. A variety of other nanoparticle-mediated drug delivery approaches have also been explored for improved Dox delivery. DNA offers many advantages relative to alternative strategies. DNA is readily biodegradable and can be used for in vivo applications without activating an immune response. Further, the use of DNA for drug delivery allows for natural combination of diverse anticancer drugs of different classes. For example, in one embodiment, the present invention allows for the inclusion of cytotoxic nucleotide analogs into ssDNA and into DNA hairpins. In an embodiment, it was also shown that duplex or hairpin DNA can be used for delivery of minor groove binding ligands. The present studies have extended this work covalent modification of DNA hairpins with simultaneous intercalation by DNA-targeting drugs, such as the Top2 poison Dox. Studies have also shown that the major groove of DNA can be used for improved drug delivery as studies have shown that Zn²⁺ complexation occurs in the major groove of FdU-substituted DNA hairpins.

Drug delivery is a multi-faceted process that involves not only improved stability in circulation but also specific uptake into targeted cells and ultimately release of drug following cell uptake. In one embodiment, the present invention shows that with the single-stranded DNA that conjugation with folic acid improves uptake into targeted cancer cells. Many cancer cells overexpress folate receptor as a consequence of increased nutrient requirements to support an elevated growth rate for the malignant phenotype. These studies show cell uptake of a 25 nucleotide DNA hairpin can be significantly enhanced into 4T1 breast cancer cells through folic acid conjugation. Importantly, while the Dox-conjugated hairpin is highly stable at physiological pH Dox release is favored at the acidic pH of endosomes following cell uptake. Dox is efficiently released from the hairpin following cell uptake and Dox retains potency as an anti-cancer drug. The results demonstrate that the DNA-conjugation strategy developed has the requisite components to be useful for Dox delivery in a clinical setting.

To the inventors knowledge this is the first report of the use of a Dox-DNA covalent conjugate to transfer Dox to DNA for potential therapeutic applications. The approach adopted has potential for greatly expanded drug delivery applications. For example it has been previously shown that FdU nucleotides can be embedded within this hairpin sequence and that the resulting hairpin is cytotoxic to prostate cancer cells. As DNA polymers containing FdU nucleotides are Top1 poisons the present system allows for creating complexes that deliver both FdU and Dox and that will simultaneously target Top1 and Top2. Simultaneous targeting of Top1 and Top2 has shown promise for clinical management of cancer although this combination displays systemic toxicities. Studies show that folic acid conjugation can be used to improve uptake for DNA hairpin conjugates into breast cancer cells and this is expected to concomitantly reduce systemic toxicities. Studies are underway to evaluate these promising concepts in drug delivery. Future studies will focus on demonstrating advantages in cellular and animal models of cancer.

The “GAA” sequence motif that promotes intramolecular DNA hairpin formation can be selectively conjugated to the Top2-poisoning anti-cancer drug Dox. The resulting conjugate is highly stable at physiological pH as a consequence of both covalent modification and intercalation of the tetracene ring system of Dox into the hairpin stem. Folic acid conjugation of the Dox-conjugated hairpin enhances uptake by 4T1 breast cancer cells. Dox is efficiently released at the acidic pH of endosomes following cell uptake demonstrating that the Dox-conjugated hairpin has both appropriate extracellular stability and intracellular lability well-suited for drug delivery applications. The DNA hairpin structural motif permits further development by inclusion of additional or alternative cytotoxic drugs, such as FdU or other cytotoxic nucleotide analogs demonstrating the multi-functional properties of DNA as a material for drug delivery science.

In an embodiment, the present invention relates to conjugates, compositions and methods using a DNA hairpin conjugate for the improved delivery of anticancer drugs. In one embodiment, the present invention relates to conjugates, compositions and methods using a DNA hairpin conjugate for the improved delivery of Doxorubicin comprising a DNA hairpin structure and Doxorubicin. In one embodiment, the Doxorubicin is covalently attached at a guanine residue in a loop region of said DNA hairpin structure.

In one embodiment, the DNA hairpin structure comprises a loop region and a stem region. In one embodiment, the stem region of the DNA hairpin structure comprises base pairs that are between about 2 and 30 nucleotides in number or between about 6 and 26 nucleotides in number, or between about 10 and 24 nucleotides in number. In one embodiment, the stem region of the DNA hairpin structure comprises 20 nucleotides.

In an embodiment, the present invention relates to Dox-hairpins using anti-folates that target one or more of folate receptor alpha and beta (e.g. lomotrexol, pemetrexed). In a variation, the present invention relates to conjugates, compositions and methods that may also use reduced folate carrier (RFC) e.g. methotrexate. It should be noted that RFC is expressed widely so may have applications in a more general manner while FRα and RR are cancer cell specific and thus, may be used to more directly target cancer cells. FRα may be more important as it relates to solid tumors. In one embodiment, the hairpin has a unique adenine site that can be used for the anti-folate targeting—for example, A13 (adenine at the 13^{th} position of the DNA hairpin structure) from SEQ ID NO: 1 (see above). It should be noted that other hairpin structures are contemplated and therefore A13 is just an example specific to SEQ ID NO: 1. In one embodiment, the hairpin loop region sequentially comprises the nucleotides GAA. In a variation, the central A of the loop region is a point of attachment for anti-folates. Thus, it is contemplated and therefore within the scope of the invention that one or more anticancer drugs can be attached to a hairpin DNA (or RNA) structure. In one embodiment, a hairpin structure may have a Dox molecule attached at one nucleotide and an anti-folate at another position. For example, with SEQ ID NO: 1, Dox may be covalently attached at G15 and an antifolate attached at A13. Accordingly, this variation would be ideally suited to treating one or more cancers that can be treated with Dox and antifolates.

In an embodiment, the stem region of the DNA hairpin structure is comprised mainly of adenines and thymines (i.e., more than half of the nucleotides are adenines and thymines). In an embodiment, the stem region of the DNA hairpin structure comprises only adenines and thymines. In an embodiment, the stem region of the DNA hairpin structure comprises 10 adenines and 10 thymines.

In an embodiment, the stem region of the DNA hairpin structure comprises a sequential 10 mer of adenines that is hydrogen bonded to a sequential 10 mer of thymines. In an embodiment, a loop region occurs between the sequential 10 mer of adenines that is hydrogen bonded to the sequential 10 mer of thymines.

In an embodiment, the stem region and/or loop region may comprise one or more of fluoropyrimidine, gemcitabine and/or AraC (cytosine arabinoside) nucleotides. In a variation, at least one of these modified nucleosides are present.

In an embodiment, the loop region comprises between about 3 and 7 nucleotides in number. In a variation, the loop region comprises 5 nucleotides in number.

In an embodiment, the loop region comprises at least two guanines. In an embodiment, the loop region comprises two guanines. In an embodiment, the two guanines are not adjacent to each other.

In an embodiment, the hairpin structure is SEQ ID NO: 1.

In an embodiment, the present invention relates to conjugates, compositions and methods wherein any of the conjugates discussed above may further comprise folic acid that is or is not covalently linked to the conjugates. In one variation the folic acid is covalently linked to the 5′ terminus of the DNA hairpin structure.

In an embodiment, the present invention relates to a method of treating cancer in an individual in need thereof comprising administering to said individual a DNA hairpin conjugate comprising a DNA hairpin structure and a cancer drug. In an embodiment, the present invention relates to a method of treating cancer in an individual in need thereof comprising administering to said individual a DNA hairpin conjugate comprising a DNA hairpin structure and Doxorubicin. In one embodiment, the Doxorubicin is covalently attached at a guanine residue in a loop region of said DNA hairpin structure.

In one embodiment, the method is used in the treatment of cancer that is prostate cancer or breast cancer. In one variation, the cancer is breast cancer.

In one embodiment, the method of the present invention uses a conjugate wherein a stem region of the DNA hairpin structure comprises base pairs that are between about 2 and 30 nucleotides in number. In a variation, the stem region of the DNA hairpin structure comprises 20 nucleotides. In a variation of the method, the stem region of the DNA hairpin structure is comprised mainly of adenines and thymines. In a variation of the method, the stem region of the DNA hairpin structure comprises only adenines and thymines. In a variation of the method, the stem region of the DNA hairpin structure comprises a sequential 10 mer of adenines that is hydrogen bonded to a sequential 10 mer of thymines.

In a variation of the method, the loop region comprises between about 3 and 7 nucleotides in number. In a variation of the method, the loop region comprises 5 nucleotides in number. In a variation of the method, the loop region comprises at least two guanines.

In a variation of the method, the conjugate or composition may further comprise folic acid. In a variation of the conjugate, composition or method, the conjugate may comprise anti-folates.

In a variation, the present invention relates to pharmaceutical compositions. The pharmaceutical composition may contain pharmaceutically acceptable salts, solvates, and prodrugs thereof, and may contain diluents, excipients, carriers, or other substances necessary to increase the bioavailability or extend the lifetime of the compounds of the present invention.

Subjects that may be treated by the compounds and compositions of the present invention include, but are not limited to, horses, cows, sheep, pigs, mice, dogs, cats, primates such as chimpanzees, gorillas, rhesus monkeys, and, humans. In an embodiment, a subject is a human in need of cancer treatment.

The pharmaceutical compositions containing a compound of the invention may be in a form suitable for injection either by itself or alternatively, using liposomes, micelles, and/or nanospheres.

In an embodiment, the compositions of the present invention may be used as injectables. The composition intended for injection may be prepared according to any known method, and such compositions may contain one or more agents selected from the group consisting of solvents, co-solvents, solubilizing agents, wetting agents, suspending agents, emulsifying agents, thickening agents, chelating agents, antioxidants, reducing agents, antimicrobial preservatives, buffers, pH adjusting agents, bulking agents, protectants, tonicity adjustors, and special additives. Moreover, other non-toxic pharmaceutically-acceptable excipients which are suitable for the manufacture of injectables may be used.

Aqueous suspensions may contain the active compounds in an admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycethanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more coloring agents.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavoring, and coloring agents may also be present.

The pharmaceutical compositions of the invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, sterile water for injection (SWFI), Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

Thus, in another embodiment, the present invention provides a pharmaceutical formulation solution comprising the conjugate(s) as described herein or a salt thereof.

A solution of the invention may be provided in a sealed container, especially one made of glass, either in a unit dosage form or in a multiple dosage form.

Any pharmaceutically acceptable salt of a compound of the conjugate(s) as described herein may be used for preparing a solution of the invention. Examples of suitable salts may be, for instance, the salts with mineral inorganic acids such as hydrochloric, hydrobromic, sulfuric, phosphoric, nitric and the like, and the salts with certain organic acids such as acetic, succinic, tartaric, ascorbic, citric, glutamic, benzoic, methanesulfonic, ethanesulfonic and the like. In an embodiment, the conjugate(s) as described herein is a hydrochloric acid salt including a mono, di, or trihydrochloride.

Any solvent which is pharmaceutically acceptable and which is able to dissolve the conjugate(s) as described herein or a pharmaceutically acceptable salt thereof may be used. The solution of the invention may also contain one or more additional components such as a co-solubilizing agent (which may be the same as a solvent), a tonicity adjustment agent, a stabilizing agent, a preservative, or mixtures thereof. Examples of solvents, co-solubilizing agents, tonicity adjustment agents, stabilizing agents and preservatives which may suitable for a solution formulation are described below.

Suitable solvents and co-solubilizing agents may include, but are not limited to, water; sterile water for injection (SWFI); physiological saline; alcohols, e.g. ethanol, benzyl alcohol and the like; glycols and polyalcohols, e.g. propyleneglycol, glycerin and the like; esters of polyalcohols, e.g. diacetine, triacetine and the like; polyglycols and polyethers, e.g. polyethyleneglycol 400, propyleneglycol methylethers and the like; dioxolanes, e.g. isopropylidenglycerin and the like; dimethylisosorbide; pyrrolidone derivatives, e.g. 2-pyrrolidone, N-methyl-2-pyrrolidone, polyvinylpyrrolidone (co-solubilizing agent only) and the like; polyoxyethylenated fatty alcohols; esters of polyoxyethylenated fatty acids; polysorbates, e.g., Tween™, polyoxyethylene derivatives of polypropyleneglycols, e.g., Pluronics™.

Suitable tonicity adjustment agents may include, but are not limited to, pharmaceutically acceptable inorganic chlorides, e.g. sodium chloride; dextrose; lactose; mannitol; sorbitol and the like.

Preservatives suitable for physiological administration may be, for instance, esters of parahydroxybenzoic acid (e.g., methyl, ethyl, propyl and butyl esters, or mixtures of them), chlorocresol and the like.

Suitable stabilizing agents include, but are not limited to, monosaccharides (e.g., galactose, fructose, and fucose), disaccharides (e.g., lactose), polysaccharides (e.g., dextran), cyclic oligosaccharides (e.g., alpha-, beta-, gamma-cyclodextrin), aliphatic polyols (e.g., mannitol, sorbitol, and thioglycerol), cyclic polyols (e.g. inositol) and organic solvents (e.g., ethyl alcohol and glycerol).

The above mentioned solvents and co-solubilizing agents, tonicity adjustment agents, stabilizing agents and preservatives can be used alone or as a mixture of two or more of them in a solution formulation.

In an embodiment, a pharmaceutical solution formulation may comprise the conjugate(s) as described herein or a pharmaceutically acceptable salt thereof, and an agent selected from the group consisting of sodium chloride solution (i.e., physiological saline), dextrose, mannitol, or sorbitol, wherein the agent is in an amount of less than or equal to 5%. The pH of such a formulation may also be adjusted to improve the storage stability using a pharmaceutically acceptable acid or base.

In the solutions of the invention the concentration of the conjugate(s) as described herein or a pharmaceutically acceptable salt thereof may be less than 100 mg/mL, or less than 50 mg/mL, or less than 10 mg/mL, or less than 10 mg/mL and greater than 0.01 mg/mL, or between 0.5 mg/mL and 5 mg/mL, or between 1 mg/mL and 3 mg/mL. In an embodiment, the concentration that is used is the ideal concentration to be sufficiently cytotoxic to the cancer cells yet limit the toxicity on other cells.

Suitable packaging for the pharmaceutical solution formulations may be all approved containers intended for parenteral use, such as plastic and glass containers, ready-to-use syringes and the like. In an embodiment, the container is a sealed glass container, e.g. a vial or an ampoule. A hermetically sealed glass vial is particularly preferred.

According to an embodiment of the present invention, there is provided, in a sealed glass container, a sterile, injectable solution comprising the conjugate(s) as described herein or a pharmaceutically acceptable salt thereof in a physiologically acceptable solvent, and which has a pH of from 2.5 to 3.5. For solution formulations, various compounds of the present invention may be more soluble or stable for longer periods in solutions at a pH lower than 6. Further, acid salts of the compounds of the present invention may be more soluble in aqueous solutions than their free base counter parts, but when the acid salts are added to aqueous solutions the pH of the solution may be too low to be suitable for administration. Thus, solution formulations having a pH above pH 4.5 may be combined prior to administration with a diluent solution of pH greater than 7 such that the pH of the combination formulation administered is pH 4.5 or higher. In one embodiment, the diluent solution comprises a pharmaceutically acceptable base such as sodium hydroxide. In another embodiment, the diluent solution is at pH of between 10 and 12. In another embodiment, the pH of the combined formulation administered is greater than 5.0. In another embodiment, the pH of the combined formulation administered is between pH 5.0 and 7.0.

The invention also provides a process for producing a sterile solution with a pH of from 2.5 to 3.5 which process comprises dissolving the conjugate(s) as described herein or a pharmaceutically acceptable salt thereof in a pharmaceutically acceptable solvent. Where a pharmaceutically acceptable acid salt of the conjugate(s) as described herein is used the pH of the solution may be adjusted using a pharmaceutically acceptable base or basic solution adding a physiologically acceptable acid or buffer to adjust the pH within a desired range. The method may further comprise passing the resulting solution through a sterilizing filter.

In an embodiment, there may be the release of conjugates based upon pH-sensitivity. This is illustrated in FIG. 20 wherein the pH sensitive release for conjugates based on carbamate linkages is shown. FIG. 20 shows the pH-sensitive release mechanism specifically for warheads containing carrier groups (here folic acid was chosen as an example) coupled via carbamate linkages. In this setup, the group cis-aconic acid is linked to the hydroxyl-modified platinum-acridine agent by using a self-immolative spacer N,N′-dimethylethylenediamine to form an amide bond. The resulting amide is readily cleaved at slightly acidic pH encountered in for example, endosomes and lysosomes.

One or more additional components such as co-solubilizing agents, tonicity adjustment agents, stabilizing agents and preservatives, for instance of the kind previously specified, may be added to the solution prior to passing the solution through the sterilizing filter.

In a further variation, the present invention contemplates combination therapies in which the compounds of the present invention can be used in conjunction with other cisplatin compounds. The efficacy of this combination therapy is likely to be enhanced because of the different mechanisms and modes of action that first generation cisplatin compounds exhibit relative to the compounds of the present invention. It is also contemplated and therefore within the scope of the invention that other anti-neoplastic agents/compounds can be used in conjunction with the compounds of the present invention. The anti-neoplastic agents/compounds that can be used with the compounds of the present invention include cytotoxic compounds as well as non-cytotoxic compounds.

Examples include anti-tumor agents such as HERCEPTIN™ (trastuzumab), RITUXAN™ (rituximab), ZEVALIN™ (ibritumomab tiuxetan), LYMPHOCIDE™ (epratuzumab), GLEEVAC™ and BEXXAR™ (iodine 131 tositumomab).

Other anti-neoplastic agents/compounds that can be used in conjunction with the compounds of the present invention include anti-angiogenic compounds such as ERBITUX™ (IMC-C225), KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as AVASTIN™ or VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as ABX-EGF (panitumumab), IRESSA™ (gefitinib), TARCEVA™ (erlotinib), anti-Ang1 and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto).

Other anti-angiogenic compounds/agents that can be used in conjunction with the compounds of the present invention include Campath, IL-8, B-FGF, Tek antagonists, anti-TWEAK agents (e.g., specifically binding antibodies or antigen binding regions, or soluble TWEAK receptor antagonists, ADAM distintegrin domain to antagonize the binding of integrin to its ligands, specifically binding anti-eph receptor and/or anti-ephrin antibodies or antigen binding regions, and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto).

Other anti-angiogenic/anti-tumor agents that can be used in conjunction with the compounds of the present invention include: SD-7784 (Pfizer, USA); cilengitide. (Merck KGaA, Germany, EPO 770622); pegaptanib octasodium, (Gilead Sciences, USA); Alphastatin, (BioActa, UK); M-PGA, (Celgene, USA); ilomastat, (Arriva, USA); emaxanib, (Pfizer, USA); vatalanib, (Novartis, Switzerland); 2-methoxyestradiol, (EntreMed, USA); TLC ELL-12, (Elan, Ireland); anecortave acetate, (Alcon, USA); alpha-D148 Mab, (Amgen, USA); CEP-7055, (Cephalon, USA); anti-Vn Mab, (Crucell, Netherlands) DAC:antiangiogenic, (ConjuChem, Canada); Angiocidin, (InKine Pharmaceutical, USA); KM-2550, (Kyowa Hakko, Japan); SU-0879, (Pfizer, USA); CGP-79787, (Novartis, Switzerland); the ARGENT technology of Ariad, USA; YIGSR-Stealth, (Johnson & Johnson, USA); fibrinogen-E fragment, (BioActa, UK); the angiogenesis inhibitors of Trigen, UK; TBC-1635, (Encysive Pharmaceuticals, USA); SC-236, (Pfizer, USA); ABT-567, (Abbott, USA); Metastatin, (EntreMed, USA); angiogenesis inhibitor, (Tripep, Sweden); maspin, (Sosei, Japan); 2-methoxyestradiol, (Oncology Sciences Corporation, USA); ER-68203-00, (WVAX, USA); Benefin, (Lane Labs, USA); Tz-93, (Tsumura, Japan); TAN-1120, (Takeda, Japan); FR-111142, (Fujisawa, Japan); platelet factor 4, (RepliGen, USA); vascular endothelial growth factor antagonist, (Borean, Denmark); bevacizumab (pINN), (Genentech, USA); XL 784, (Exelixis, USA); XL 647, (Exelixis, USA); MAb, alpha5beta3 integrin, second generation, (Applied Molecular Evolution, USA and MedImmune, USA); gene therapy, retinopathy, (Oxford BioMedica, UK); enzastaurin hydrochloride (USAN), (Lilly, USA); CEP 7055, (Cephalon, USA and Sanofi-Synthelabo, France); BC 1, (Genoa Institute of Cancer Research, Italy); angiogenesis inhibitor, (Alchemia, Australia); VEGF antagonist, (Regeneron, USA); rBPI 21 and BPI-derived antiangiogenic, (XOMA, USA); PI 88, (Progen, Australia); cilengitide (pINN), (Merck KGaA, German; Munich Technical University, Germany, Scripps Clinic and Research Foundation, USA); cetuximab (INN), (Aventis, France); AVE 8062, (Ajinomoto, Japan); AS 1404, (Cancer Research Laboratory, New Zealand); SG292, (Telios, USA); Endostatin, (Boston Children's Hospital, USA); ATN 161, (Attenuon, USA); ANGIOSTATIN, (Boston Children's Hospital, USA); 2-methoxyestradiol, (Boston Children's Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenesis, (EntreMed, USA); urokinase plasminogen activator inhibitor, (Dendreon, USA); oglufanide (pINN), (Melmotte, USA); HIF-1alfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol, (EntreMed, USA); anginex, (Maastricht University, Netherlands, and Minnesota University, USA); ABT 510, (Abbott, USA); AAL 993, (Novartis, Switzerland); VEGI, (ProteomTech, USA); tumor necrosis factor-alpha inhibitors, (National Institute on Aging, USA); SU 11248, (Pfizer, USA and SUGEN USA); ABT 518, (Abbott, USA); YH16, (Yantai Rongchang, China); S-3APG, (Boston Children's Hospital, USA and EntreMed, USA); MAb, KDR, (ImClone Systems, USA); MAb, alpha5 beta1, (Protein Design, USA); KDR kinase inhibitor, (Celltech Group, UK, and Johnson & Johnson, USA); GFB 116, (South Florida University, USA and Yale University, USA); CS 706, (Sankyo, Japan); combretastatin A4 prodrugs, (Arizona State University, USA); chondroitinase AC, (IBEX, Canada); BAY RES 2690, (Bayer, Germany); AGM 1470, (Harvard University, USA, Takeda, Japan, and TAP, USA); AG 13925, (Agouron, USA); Tetrathiomolybdate, (University of Michigan, USA); GCS 100, (Wayne State University, USA) CV 247, (Ivy Medical, UK); CKD 732, (Chong Kun Dang, South Korea); MAb, vascular endothelium growth factor, (Xenova, UK); irsogladine (INN), (Nippon Shinyaku, Japan); RG 13577, (Aventis, France); WX 360, (Wilex, Germany); squalamine (pIN), (Genaera, USA); RPI 4610, (Sima, USA); heparanase inhibitors, (InSight, Israel); KL 3106, (Kolon, South Korea); Honokiol, (Emory University, USA); ZK CDK, (Schering AG, Germany); ZK Angio, (Schering AG, Germany); ZK 229561, (Novartis, Switzerland, and Schering AG, Germany); XMP 300, (XOMA, USA); VGA 1102, (Taisho, Japan); VEGF receptor modulators, (Pharmacopeia, USA); VE-cadherin-2 antagonists, (ImClone Systems, USA); Vasostatin, (National Institutes of Health, USA); vaccine, Flk-1, (ImClone Systems, USA); TZ 93, (Tsumura, Japan); TumStatin, (Beth Israel Hospital, USA); truncated soluble FLT 1 (vascular endothelial growth factor receptor 1), (Merck & Co, USA); Tie-2 ligands, (Regeneron, USA); and, thrombospondin 1 inhibitor, (Allegheny Health, Education and Research Foundation, USA).

It is contemplated and therefore within the scope of the invention that the compounds of the present invention can be modified to target specific receptors or cancer cells or can be modified so that they can survive various in vivo environments. In a variation, the conjugates, compositions, and methods of the present invention can be used against solid tumors, cell lines, and cell line tissue that demonstrate upregulated nucleotide excision repair and other upregulated resistance mechanisms.

The following references are incorporated by reference in their entireties:
(1) Elliott, P. (2006) Pathogenesis of cardiotoxicity induced by anthracyclines. Semin. Oncol. 33(3Suppl8), S2-7.
(2) Sordet O., Khan Q. A., Kohn K. W., and Pommier Y. (2003) Apoptosis Induced by Topoisomerase Inhibitors. Current Medicinal Chemistry—Anti-Cancer Agents 3, 271-290.
(3) Zhang, S., Liu, X., Bawa-Khalfe, T., Lu, L.-S., Lyu, Y. L., Liu, L. F., and Yeh, E. T. H. (2012) Identification of the molecular basis of doxorubicin-induced cardiotoxicity. Nat. Med. 18, 1639-1642.
(4) Liao, Z.-Y., Sordet, O., Zhang, H.-L., Kohlhagen, G., Antony, S., Gmeiner, W. H., and Pommier, Y. (2005) A novel polypyrimidine antitumor agent FdUMP[10] induces thymineless death with topoisomerase I-DNA complexes. Cancer Res. 65, 4844-4851.
(5) Pardee, T. S., Gomes, E., Jennings-Gee, J., Caudell, D., and Gmeiner, W. H. (2012) Unique dual targeting of thymidylate synthase and topoisomerase1 by FdUMP[10] results in high efficacy against AML and low toxicity. Blood 119, 3561-3570.
(6) Ghosh, S., Salsbury, F. R., Jr, Horita, D. A., and Gmeiner, W. H. (2011) Zn2+ selectively stabilizes FdU-substituted DNA through a unique major groove binding motif. Nucleic Acids Rev. 39, 4490-4498.
(7) Ghosh, S., Salsbury, F. R., Jr, Horita, D. A., and Gmeiner, W. H. (2013) Cooperative stabilization of Zn(2+):DNA complexes through netropsin binding in the minor groove of FdU-substituted DNA. J. Biomol. Struct. Dyn. 31, 1301-1310.
(8) Wang, A. H., Gao, Y. G., Liaw, Y. C., and Li, Y. K. (1991) Formaldehyde cross-links daunorubicin and DNA efficiently: HPLC and X-ray diffraction studies. Biochemistry 30, 3812-3815.
(9) Trouet, A., and Jolles, G. (1984) Targeting of Daunorubicin by Association With DNA or Proteins: A Review. Semin Oncol 11, (4Suppl3) 64-72.
(10) Zeman, S. M., Phillips, D. R., and Crothers, D. M. (1998) Characterization of covalent Adriamycin-DNA adducts. Proc. Natl. Acad. Sci. U.S.A. 95, 11561-11565.
(11) Cutts, S. M., Parker, B. S., Swift, L. P., Kimura, K. I., and Phillips, D. R. (2000) Structural requirements for the formation of anthracycline-DNA adducts. Anticancer Drug Des. 15, 373-386.
(12) Swift, L. P., Cutts, S. M., Rephaeli, A., Nudelman, A., and Phillips, D. R. (2003) Activation of adriamycin by the pH-dependent formaldehyde-releasing prodrug hexamethylenetetramine. Mol. Cancer Ther. 2, 189-198.
(13) Post, G. C., Barthel, B. L., Burkhart, D. J., Hagadom, J. R., and Koch, T. H. (2005) Doxazolidine, a proposed active metabolite of doxorubicin that cross-links DNA. J. Med. Chem. 48, 7648-7657.
(14) Phillips, D. R., White, R. J., and Cullinane, C. (1989) DNA sequence-specific adducts of adriamycin and mitomycin C. FEBS Lett. 246, 233-240.
(15) Lee, R. J., and Low, P. S. (1994) Delivery of liposomes into cultured KB cells via folate receptor-mediated endocytosis. J. Biol. Chem. 269, 3198-3204.
(16) Zhang, H., Cai, Z., Sun, Y., Yu, F., Chen, Y., and Sun, B. (2012) Folate-conjugated β-cyclodextrin from click chemistry strategy and for tumor-targeted drug delivery. J. Biomed. Mater. Res. A 100, 2441-2449.
(17) Piotto, M., Saudek, V., and Sklenár, V. (1992) Gradient-tailored excitation for single-quantum NMR spectroscopy of aqueous solutions. J. Biomol. NMR 2, 661-665.
(18) Delaglio, F., Grzesiek, S., Vuister, G. W., Zhu, G., Pfeifer, J., and Bax, A. (1995) NMRPipe: a multidimensional spectral processing system based on UNIX pipes. J. Biomol. NMR 6, 277-293.
(19) Ulyanov, N. B., Bauer, W. R., and James, T. L. (2002) High-resolution NMR structure of an AT-rich DNA sequence. J. Biomol. NMR 22, 265-280.
(20) Humphrey, W., Dalke, A., and Schulten, K. (1996) VMD: visual molecular dynamics., J. Mol. Graph. 14, 33-38.
(21) Stewart, J. J. P. (2007) Optimization of parameters for semiempirical methods V: Modification of NDDO approximations and application to 70 elements. J. Mol. Model. 13, 1173-1213.
(22) Bouaziz, S., Kettani, A., and Patel, D. J. (1998) A K cation-induced conformational switch within a loop spanning segment of a DNA quadruplex containing G-G-G-C repeats., Journal of Molecular Biology 282, 637-652.
(23) Liu, J., Kolar, C., Lawson, T. A., and Gmeiner, W. H. (2001) Targeted drug delivery to chemoresistant cells: folic acid derivatization of FdUMP[10] enhances cytotoxicity toward 5-FU-resistant human colorectal tumor cells. J. Org. Chem. 66, 5655-5663.
(24) (1) Bagalkot, V., Lee, I.-H., Yu, M. K., Lee, E., Park, S., Lee, J.-H., and Jon, S. (2009) A combined chemoimmunotherapy approach using a plasmid-doxorubicin complex. Mol. Pharm. 6, 1019-1028.
(25) O'Brien, M. E. R., Wigler, N., Inbar, M., Rosso, R., Grischke, E., Santoro, A., Catane, R., Kieback, D. G., Tomczak, P., Ackland, S. P., Orlandi, F., Mellars, L., Alland, L., Tendler, C., and CAELYX Breast Cancer Study Group. (2004) Reduced cardiotoxicity and comparable efficacy in a phase III trial of pegylated liposomal doxorubicin HCl (CAELYX/Doxil) versus conventional doxorubicin for first-line treatment of metastatic breast cancer. Ann. Oncol. 15, 440-449.
(26) Boyacioglu, O., Stuart, C. H., Kulik, G., and Gmeiner, W. H. (2013) Dimeric DNA Aptamer Complexes for High-capacity-targeted Drug Delivery Using pH-sensitive Covalent Linkages. Mol. Ther. Nucleic Acids 2, e107.
(27) Janes, K. A., Fresneau, M. P., Marazuela, A., Fabra, A., and Alonso, M. J. (2001) Chitosan nanoparticles as delivery systems for doxorubicin. J. Control Release 73, 255-267.
(28) Chang, Y., Liu, N., Chen, L., Meng, X., Liu, Y., Li, Y., and Wang, J. (2012) Synthesis and characterization of DOX-conjugated dendrimer-modified magnetic iron oxide conjugates for magnetic resonance imaging, targeting, and drug delivery. Journal of Materials Chemistry 22, 9594.
(29) Russell-Jones, G., McTavish, K., McEwan, J., Rice, J., and Nowotnik, D. (2004) Vitamin-mediated targeting as a potential mechanism to increase drug uptake by tumours. J. Inorg. Biochem. 98, 1625-1633.
(30) Eder, J. P., Chan, V., Wong, J., Wong, Y. W., Ara, G., Northey, D., Rizvi, N., and Teicher, B. A. (1998) Sequence effect of irinotecan (CPT-11) and topoisomerase II inhibitors in vivo. Cancer Chemother. Pharmacol. 42, 327-335.
(31) Morgensztern, D., Baggstrom, M. Q., Pillot, G., Tan, B., Fracasso, P., Suresh, R., Wildi, J., and Govindan, R. (2009) A phase I study of pegylated liposomal doxorubicin and irinotecan in patients with solid tumors. Chemotherapy 55, 441-445.

It is contemplated and therefore within the scope of the present invention that any feature that is described above can be combined with any other feature that is described above. When conjugates are discussed, it should be understood that those conjugates are contemplated as being parts of compositions and/or methods of treatment and/or methods of making. Moreover, it should be understood that the present invention contemplates minor modifications that can be made to the compounds, conjugates, compositions and methods of the present invention. In any event, the present invention is defined by the below claims.

We claim:

1. A DNA hairpin conjugate for the improved delivery of Doxorubicin and/or other anti-cancer drugs comprising a DNA hairpin structure and Doxorubicin wherein said Doxorubicin and/or other anti-cancer drug is covalently attached at a guanine residue in a loop region of said DNA hairpin structure.

2. The conjugate of claim 1, wherein a stem region of the DNA hairpin structure comprises base pairs that are between about 2 and 30 nucleotides in number.

3. The conjugate of claim 2, wherein the stem region of the DNA hairpin structure comprises 20 nucleotides.

4. The conjugate of claim 3, wherein the stem region of the DNA hairpin structure is comprised mainly of adenines and thymines.

5. The conjugate of claim 4, wherein the stem region of the DNA hairpin structure comprises only adenines and thymines.

6. The conjugate of claim 4, wherein the stem region of the DNA hairpin structure comprises a sequential 10 mer of adenines that is hydrogen bonded to a sequential 10 mer of thymines.

7. The conjugate of claim 6, wherein the loop region comprises between about 3 and 7 nucleotides in number.

8. The conjugate of claim 7, wherein the loop region comprises 5 nucleotides in number.

9. The conjugate of claim 8, wherein the loop region comprises at least two guanines.

10. The conjugate of claim 1, further comprising folic acid.

11. A method of treating cancer in an individual in need thereof comprising administering to said individual a DNA hairpin conjugate comprising a DNA hairpin structure and Doxorubicin or other anti-cancer drugs wherein said Doxorubicin and/or other anti-cancer drugs is covalently attached at a guanine residue in a loop region of said DNA hairpin structure.

12. The method of claim 11, wherein the cancer is breast cancer.

13. The method of claim 11, wherein a stem region of the DNA hairpin structure comprises base pairs that are between about 2 and 30 nucleotides in number.

14. The method of claim 13, wherein the stem region of the DNA hairpin structure comprises 20 nucleotides.

15. The method of claim 14, wherein the stem region of the DNA hairpin structure is comprised mainly of adenines and thymines.

16. The method of claim 15, wherein the stem region of the DNA hairpin structure comprises only adenines and thymines.

17. The method of claim 16, wherein the stem region of the DNA hairpin structure comprises a sequential 10 mer of adenines that is hydrogen bonded to a sequential 10 mer of thymines.

18. The method of claim 11, wherein the loop region comprises between about 3 and 7 nucleotides in number.

19. The method of claim 18, wherein the loop region comprises 5 nucleotides in number.

20. The method of claim 19, wherein the loop region comprises at least two guanines.

21. The method of claim 11, further comprising folic acid.

22. The conjugate of claim 1, wherein the conjugate further comprises one or more anti-folates.

23. The conjugate of claim 22, wherein the conjugate comprises both Doxorubicin and an anti-folate covalently attached to the DNA hairpin structure.


SEQUENCE LISTING (1 sequence)

SEQ ID NO: 1 - DNA, 25 bp, Artificial Sequence
  synthetic
aaaaaaaaaacgaagtttttttttt